(19) United States
(12) Patent Application Publication
(10) Pub. No.: US 2023/0108831 A1
(43) Pub. Date: Apr. 6, 2023

(54) Scaffold-Free Nerve Conduit

(71) Applicant: University of Pittsburgh - Of the Commonwealth System of Higher Education, Pittsburgh, PA (US)

(72) Inventor: Fatima Syed-Picard, Pittsburgh, PA (US)

(21) Appl. No.: 17/909,263

(22) PCT Filed: Mar. 5, 2021
(86) PCT No.: PCT/US2021/021124

§ 371 Date: Sep. 2, 2022

Related U.S. Application Data
(60) Provisional application No. 62/986,116, filed on Mar. 6, 2020.

(51) Int. Cl. A61L 27/36; A61L 27/38

(52) U.S. Cl. CPC A61L 27/3675; A61L 27/383; A61L 27/3691; A61L 27/3804; A61L 27/3834; A61L 27/3895; A61L 2300/252

(57) ABSTRACT

A scaffold-free nerve conduit and a method of making the scaffold-free nerve conduit are provided. A nerve-repair method using the scaffold-free nerve conduit also is provided.

### CROSS REFERENCE TO RELATED APPLICATIONS

This application is the United States national phase of International Application No. PCT/US2021/021124 filed Mar. 5, 2021, and claims priority to U.S. Provisional Pat. Application No. 62/986,116 filed Mar. 6, 2020, the disclosures of which are hereby incorporated by reference in their entirety.

### BACKGROUND OF THE INVENTION

### Field of the Invention

Provided herein are scaffold-free nerve conduits, methods of making scaffold-free nerve conduits, and methods of using scaffold-free nerve conduits for nerve repair.

### Description of Related Art

Nerve defects, such as facial nerve defects, may result from trauma, tumor ablation, and iatrogenic surgical injury. The current gold standard for peripheral nerve repair is to perform direct end-to-end reconnection in small nerve gaps (e.g., < 1 cm) or to replace damaged tissue with an autograft in larger defects. In both of these cases, nerve regeneration can take up to 18 months and complete functional recovery may not be achieved. Additionally, in the case of autograft procedures, a functional nerve at a harvest site must be sacrificed, resulting in sensory loss. Decellularized allografts and synthetic polymeric conduits are marketed for nerve repair by several companies, but these lack important biological cues (e.g., cells, matrix, and growth factors) that are known to accelerate nerve repair and regeneration.

Nerve conduits providing biological cues and supporting directional neurite extension are needed.

### SUMMARY OF THE INVENTION

A method of preparing a nerve conduit for use in repairing nerve damage or a nerve defect in a patient is provided. The method comprising: culturing a population of cells comprising a stem cell to confluence, or over-confluence, on a substrate comprising microgrooves aligned in a first direction, to produce a sheet of cells aligned in the first direction; separating the aligned sheet of cells from the substrate; rolling the sheet of cells in a second direction to form a roll of cells having a longitudinal axis extending in the first direction relative to the sheet of cells; and culturing the roll of cells to fuse the rolled cell sheet to form a cylindrical cell structure, e.g., by adhesion and/or remodeling of adjacent layers of cells in the cylindrical roll of cells, wherein the cells produce, or are induced or are differentiated to produce, a neurotrophic factor.

A method of preparing a nerve conduit for use in repairing nerve damage or a nerve defect in a patient is provided. The method comprising: culturing a population of cells comprising a stem cell to confluence, or over-confluence, on a substrate, to produce a sheet of cells; separating the sheet of cells from the substrate; and culturing the sheet of cells anchored at two or more points to substantially align the cells in the sheet of cells, wherein the cells produce, or are induced or differentiated to produce, a neurotrophic factor.

A tissue culture vessel is provided. The vessel comprising a sheet of cells anchored at two or more points along its length in culture medium, wherein the sheet of cells is prepared by: culturing a population of cells comprising a stem cell to confluence, or over-confluence, on a substrate, to produce a sheet of cells; separating the sheet of cells from the substrate; and culturing the sheet of cells anchored at two or more points to substantially align the cells in the sheet of cells, wherein the cells produce, or are induced or differentiated to produce, a neurotrophic factor.

A tissue culture vessel is provided. The vessel comprising a fused, cylindrical cell structure prepared by: culturing a population of cells comprising a stem cell to confluence, or over-confluence, on a substrate comprising microgrooves aligned in a first direction, to produce a sheet of cells aligned in the first direction; separating the aligned sheet of cells from the substrate; and rolling the sheet of cells in a second direction to form a roll of cells having a longitudinal axis extending in the first direction relative to the sheet of cells, wherein the cells produce, or are induced or differentiated to produce, a neurotrophic factor.

According to a first non-limiting aspect or embodiment, provided is a method of preparing a nerve conduit for use in repairing nerve damage or a nerve defect in a patient, comprising:
culturing a population of cells comprising a stem cell to confluence, or over-confluence, on a substrate comprising microgrooves aligned in a first direction, to produce a sheet of cells aligned in the first direction;
separating the aligned sheet of cells from the substrate;
rolling the sheet of cells in a second direction to form a roll of cells having a longitudinal axis extending in the first direction relative to the sheet of cells; and
culturing the roll of cells to fuse the rolled cell sheet to form a cylindrical cell structure, e.g., by adhesion and/or remodeling of adjacent layers of cells in the cylindrical roll of cells,
wherein the cells produce, or are induced or are differentiated to produce, a neurotrophic factor.

According to a second non-limiting aspect or embodiment, provided is a method according the first non-limiting aspect or embodiment, wherein the cells are obtained or derived from neural crest tissue and comprise neural crest stem cells

According to a third non-limiting aspect or embodiment, provided is a method according the first non-limiting aspect or embodiment, wherein the cells are dental pulp cells or are obtained from a population of dental pulp cells.

According to a fourth non-limiting aspect or embodiment, provided is a method according the first non-limiting aspect or embodiment, wherein the cells produce one or more neurotrophic factors.

According to a fifth non-limiting aspect or embodiment, provided is a method according the fourth non-limiting aspect or embodiment, wherein the one or more neurotrophic factors is brain-derived growth factor (BDNF), glial-cell derived neurotrophic factor (GDNF), or neurotrophin-3 (NT-3).

According to a sixth non-limiting aspect or embodiment, provided is a method according the fourth non-limiting aspect or embodiment, wherein the cells produce BDNF, GDNF, and NT-3.

According to a seventh non-limiting aspect or embodiment, provided is a method according the first non-limiting aspect or embodiment, further comprising, after the sheet of cells is produced, differentiating the cells to a cell type that produces one or more neurotrophic factors.

According to an eighth non-limiting aspect or embodiment, provided is a method according the seventh non-limiting aspect or embodiment, wherein the cells are differentiated into a Schwann cell-like phenotype.

According to a ninth non-limiting aspect or embodiment, provided is a method according the first non-limiting aspect or embodiment, wherein the stem cell is a mesenchymal stem cell.

According to a tenth non-limiting aspect or embodiment, provided is a method according to the first non-limiting aspect or embodiment, wherein the cells comprise adipose-derived stem cells, umbilical cord stem cells, or bone marrow stem cells.

According to a eleventh non-limiting aspect or embodiment, provided is a method according to the first non-limiting aspect or embodiment, wherein the cells comprise induced pluripotent stem cells.

According to a twelfth non-limiting aspect or embodiment, provided is a method according to any one of the first through eleventh aspect or embodiment, wherein the microgrooved substrate comprises linear grooves oriented in the same direction or substantially in the same direction, or anisotropic grooves, such as parallel grooves.

According to a thirteenth non-limiting aspect or embodiment, provided is a method according to any one of the first through twelfth aspect or embodiment, wherein the grooves of the microgrooved substrate are less than 250 microns (µ), less than 100 µ, less than 50 µ, less than 25 µ, or less than 10 µ in width, such as ranging from 1 µ to 10 µ in width.

According to a fourteenth non-limiting aspect or embodiment, provided is a method of preparing a nerve conduit for use in repairing nerve damage or a nerve defect in a patient, comprising:
culturing a population of cells comprising a stem cell to confluence, or over-confluence, on a substrate, to produce a sheet of cells;
separating the sheet of cells from the substrate; and
culturing the sheet of cells anchored at two or more points to substantially align the cells in the sheet of cells,
wherein the cells produce, or are induced or differentiated to produce, a neurotrophic factor.

According to a fifteenth non-limiting aspect or embodiment, provided is a method according to the fourteenth non-limiting aspect or embodiment, wherein, prior to culturing the anchored sheet of cells, rolling the sheet of cells to form a roll of cells, culturing the rolled sheet of cells anchored at two or more points along the length, e.g., along a longitudinal axis of the rolled sheet of cells, to fuse the rolled sheet of cells to form a cylindrical cell structure, e.g., by adhesion and/or remodeling of adjacent layers of cells in the cylindrical roll of cells, and to substantially align the cells in the rolled sheet of cells lengthwise in the rolled sheet of cells, or parallel to the longitudinal axis of the rolled sheet of cells.

According to a sixteenth non-limiting aspect or embodiment, provided is a method according to the fourteenth or fifteenth non-limiting aspect or embodiment, wherein the cells are obtained or derived from neural crest tissue and comprise neural crest stem cells.

According to a seventeenth non-limiting aspect or embodiment, provided is a method according to the fourteenth or fifteenth non-limiting aspect or embodiment, wherein the cells are dental pulp cells or are obtained from a population of dental pulp cells.

According to an eighteenth non-limiting aspect or embodiment, provided is a method according to the fourteenth or fifteenth non-limiting aspect or embodiment, wherein the cells produce one or more neurotrophic factors.

According to a nineteenth non-limiting aspect or embodiment, provided is a method according to the eighteenth non-limiting aspect or embodiment, wherein the one or more neurotrophic factors is brain-derived growth factor (BDNF), glial-cell derived neurotrophic factor (GDNF), or neurotrophin-3 (NT-3).

According to a twentieth non-limiting aspect or embodiment, provided is a method according to the eighteenth non-limiting aspect or embodiment, wherein the cells produce BDNF, GDNF, and NT-3.

According to a twenty-first non-limiting aspect or embodiment, provided is a method according to the fourteenth or fifteenth non-limiting aspect or embodiment, further comprising, after the sheet of cells is produced, differentiating the cells to a cell type that produces one or more neurotrophic factors.

According to a twenty-second non-limiting aspect or embodiment, provided is a method according to the twenty-first non-limiting aspect or embodiment, wherein the cells are differentiated into a Schwann cell-like phenotype.

According to a twenty-third non-limiting aspect or embodiment, provided is a method according to the fourteenth or fifteenth non-limiting aspect or embodiment, wherein the stem cell is a mesenchymal stem cell.

According to a twenty-fourth non-limiting aspect or embodiment, provided is a method according to the fourteenth or fifteenth non-limiting aspect or embodiment, wherein the cells comprise adipose-derived stem cells, umbilical cord stem cells, or bone marrow stem cells.

According to a twenty-fifth non-limiting aspect or embodiment, provided is a method according to the fourteenth or fifteenth non-limiting aspect or embodiment, wherein the cells comprise induced pluripotent stem cells.

According to a twenty-sixth non-limiting aspect or embodiment, provided is a fused, cylindrical cell structure prepared according to the method of any one of the first through twenty-fifth aspect or embodiment.

According to a twenty-seventh non-limiting aspect or embodiment, provided is a method of repairing nerve damage or a nerve defect in a patient, comprising implanting the fused, cylindrical cell structure according to the twenty-sixth aspect or embodiment, at a site of nerve damage or nerve defect in the patient.

According to a twenty-eighth non-limiting aspect or embodiment, provided is a method according to the twenty-seventh non-limiting aspect or embodiment, wherein the nerve damage or nerve defect in the patient is damage to a facial nerve or a facial nerve defect.

According to a twenty-ninth non-limiting aspect or embodiment, provided is a tissue culture vessel comprising a sheet of cells anchored at two or more points along its length in culture medium, wherein the sheet of cells is prepared by the method according to any one of the fourteenth through twenty-fifth aspect or embodiment.

According to a thirtieth non-limiting aspect or embodiment, provided is a tissue culture vessel, comprising a fused, cylindrical cell structure prepared by the method according to any one of the first through thirteenth aspect or embodiment.

According to a thirty-first non-limiting aspect or embodiment, provided is a tissue culture vessel according to the twenty-ninth or thirtieth non-limiting aspect or embodiment, wherein the cells are obtained or derived from neural crest tissue and comprise neural crest stem cells.

According to a thirty-second non-limiting aspect or embodiment, provided is a tissue culture vessel according to the twenty-ninth or thirtieth non-limiting aspect or embodiment, wherein the cells are dental pulp cells or are obtained from a population of dental pulp cells.

According to a thirty-third non-limiting aspect or embodiment, provided is a tissue culture vessel according to the twenty-ninth or thirtieth non-limiting aspect or embodiment, wherein the cells produce one or more neurotrophic factors.

According to a thirty-fourth non-limiting aspect or embodiment, provided is a tissue culture vessel according to the thirty-third non-limiting aspect or embodiment, wherein the one or more neurotrophic factors is brain-derived growth factor (BDNF), glial-cell derived neurotrophic factor (GDNF), or neurotrophin-3 (NT-3).

According to a thirty-fifth non-limiting aspect or embodiment, provided is a tissue culture vessel according to the thirty-third non-limiting aspect or embodiment, wherein the cells produce BDNF, GDNF, and NT-3.

According to a thirty-sixth non-limiting aspect or embodiment, provided is a tissue culture vessel according to the twenty-ninth or thirtieth non-limiting aspect or embodiment, further comprising, after the sheet of cells is produced, differentiating the cells to a cell type that produces one or more neurotrophic factors.

According to a thirty-seventh non-limiting aspect or embodiment, provided is a tissue culture vessel according to the thirty-sixth non-limiting aspect or embodiment, wherein the cells are differentiated into a Schwann cell-like phenotype.

According to a thirty-eighth non-limiting aspect or embodiment, provided is a tissue culture vessel according to the twenty-ninth or thirtieth non-limiting aspect or embodiment.

According to a thirty-ninth non-limiting aspect or embodiment, provided is a tissue culture vessel according to the twenty-ninth or thirtieth non-limiting aspect or embodiment, wherein the cells comprise adipose-derived stem cells, umbilical cord stem cells, or bone marrow stem cells, wherein the stem cell is a mesenchymal stem cell.

### BRIEF DESCRIPTION OF THE DRAWINGS

FIGS. 1A and 1B provide schematic elevated and end views, respectively of an exemplary, rolled tissue sheet as described herein.

FIGS. 2A-2C provide schematic views of an exemplary culture vessel (FIG. 2A) and the culture vessel in use (FIGS. 2B and 2C), including culture medium and an anchored (pinned) rolled cell sheet.

FIG. 3 schematically depicts an exemplary method of preparing a nerve conduit as described herein.

FIG. 4 provides a schematic illustration of the methods described in Example 1.

FIG. 5 provides photomicrographs of the cross-hatched and linear substrates described in Example 1. Scale bars = 50 µm for en face and 100 µm for cross-sections.

FIGS. 6A and 6B. Phase contrast and fluorescence staining with phalloidin (actin cytoskeleton) and against collagen I showed that the DPCs and their ECM aligned with the parallel grooves (FIG. 6A); Scale bars = 100 µm. Quantification of cell alignment validated that DPCs had increased alignment on linear grooves (FIG. 6B); Scale bars = 100 µm. The angle of nuclear alignment was quantified by subtracting the angle of the major axis of the cell nuclei from the angle of the PDMS topography.

FIG. 7 provides graphs showing quantification of neurotrophic factor (NTF) genes (Brain-derived neurotrophic factor (BDNF), Glial cell line-derived neurotrophic factor (GDNF), and Neurotrophin-3 (NT-3)) and protein expression from RT-PCR and ELISA, respectively, indicated that linearly aligned DPCs expressed similar levels of NTFs than un-aligned DPCs. Therefore, alignment of DPCs had no significant effect on NTF expression.

FIG. 8 provides photomicrographs showing staining of neuronally pre-differentiated SH-SY5Y neuroblastoma cells grown on DPC sheets as described in Example 1. The cells extend neurites that are oriented in the direction of the aligned ECM when cultured on DPC sheets formed on a grooved substrate. Neurites are not aligned when cultured on DPC sheets formed on flat or cross-hatch substrates.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values.

As used herein, “a” and “an” refer to one or more.

The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” those stated elements or steps. For definitions provided herein, those definitions refer to word forms, cognates and grammatical variants of those words or phrases.

As used herein, the terms “patient” or “subject” refer to members of the animal kingdom including but not limited to human beings and “mammal” refers to all mammals, including, but not limited to human beings.

As used herein, “treatment” or “treating” of a wound or defect means administration to a patient by any suitable dosage regimen, procedure and/or administration route of a composition, device or structure with the object of achieving a desirable clinical/medical end-point, including, for example, attracting progenitor cells, healing a wound, correcting a defect, causing neurite outgrowth, or repairing a nerve.

As used herein, the terms “cell” and “cells” refer to any types of cells from any animal, such as, without limitation, rat, mouse, monkey, and human. For example and without limitation, cells can be progenitor cells, e.g., pluripotent cells, including stem cells, induced pluripotent stem cells, multipotent cells, or differentiated cells, such as endothelial cells and smooth muscle cells. “Cells” also includes populations of cells, such as, for example, a population of cells produced by culturing dental pulp or cells from other neural crest-derived tissue. In certain aspects, cells for medical procedures can be obtained from the patient for autologous procedures, or from other donors for allogeneic procedures, or from xenogeneic sources.

Provided herein is a scaffold-free cell structure, such as a nerve conduit, comprising cells, such as neural crest-derived cells or dental pulp cells, that naturally express neurotrophic factors, or cells that produce neurotrophic factors upon induction of expression of those factors or differentiation of the cells to a cell type that produces neurotrophic factors, such as a Schwann cell, or a Schwann cell-like phenotype. Cell structures can be placed adjacent to a neuron to induce neurite outgrowth from the neuron. For example, nerve conduits can be placed within a gap in a peripheral nerve, optionally sutured or anastomosed in place, by standard surgical methods to accelerate healing and enhance motor recovery.

Neurotrophic factors (NTF) are proteins known to enhance axon regeneration and growth. As an example, neural crest-derived tissue, for example, dental pulp tissue, contains a population of stem/progenitor cells (e.g., dental pulp stem cells, DPSCs) that secrete NTFs, a characteristic likely due to their neural crest origin. Furthermore, those cells are easily accessible from autologous sources. Neural crest-derived cell sheets, and nerve conduits as described herein express NTFs including brain-derived neurotrophic factor (BDNF), glial cell line-derived neurotrophic factors (GDNF) and neurotrophin-3 (NT-3), and will accelerate repair of damaged nerves and improve functional recovery.

Cell populations comprising stem cells, e.g., mesenchymal stem cells, may be used in the methods described herein (see, e.g., Samsonraj RM, et al., Concise Review: Multifaceted Characterization of Human Mesenchymal Stem Cells for Use in Regenerative Medicine. Stem Cells Transl Med. 2017 Dec;6(12):2173-2185). A stem cell may be defined as a totipotent, pluripotent, or multipotent of a multicellular organism from which certain other kinds of cells arise by differentiation. Stem cells are generally capable of giving rise to indefinitely more cells of the same type in cell culture. Stem cells may be characterized as totipotent, pluripotent, or multipotent, depending on their source. Stem cells may naturally produce neurotrophic factors, may be manipulated or induced to produce neurotrophic factors, e.g., by introducing a gene into the cells for expression of one or more neurotrophic factors, or may be differentiated into cell populations that produce neurotrophic factors (e.g., Schwann cell-like cells), and can find use in the methods and nerve conduits described herein. As such, the cells useful in the present methods and structures are populations of cells comprising cells that may be totipotent, pluripotent, or multipotent, which produce neurotrophic factors, or which are capable of producing neurotrophic factors on differentiation or induction. Although obtainable from many tissue sources, non-limiting examples of tissue sources for cell populations comprising stem cells include umbilical cord stem cells (including umbilical cord blood, umbilical cord matrix, Wharton’s jelly, etc. (see, e.g., Weiss ML, et al., Stem cells in the umbilical cord. Stem Cell Rev. 2006;2(2):155-162), adipose tissue, bone marrow, perivascular cells e.g., pericytes (see, e.g., Avolio E, Alvino VV, Ghorbel MT, Campagnolo P. Perivascular cells and tissue engineering: Current applications and untapped potential. Pharmacol Ther. 2017;171:83-92), and induced pluripotent stem cells (iPSCs, see, e.g., Yamanaka S. Induced pluripotent stem cells: past, present, and future. Cell Stem Cell. 2012 Jun 14;10(6):678-684 and Kim, HS., et al., Directly induced human Schwann cell precursors as a valuable source of Schwann cells. Stem Cell Res Ther 11, 257 (2020)). Cell populations useful in the present methods and nerve conduits may be enriched for stem cells by any useful method, including cell separation and cell sorting techniques and cell culturing techniques, as are broadly-known in the stem cell field. Methods of generation of useful iPSCs are also broadly-known. Cell populations comprising stem cells may be cryogenically preserved, e.g., in a tissue bank in which a patient’s (autologous) tissue or stem cells are stored for later retrieval.

Cells useful for nerve tissue repair can include cell populations obtained from culturing cells obtained from neural crest-derived tissue (tissue arising from the neural crest), which, among other tissues, include many of the skeletal and connective tissue of the head, or the cranial mesenchyme, such as cartilage, bone, cranial neurons, glia, and connective tissues of the face. Neural crest cells enter the pharyngeal arches and pouches to give rise to thymic cells, odontoblasts of the tooth primordia, and the bones of middle ear and jaw. Neural crest tissue includes multipotent cells referred to as neural crest stem cells (See, e.g., Achilleos, A., et al., Neural crest stem cells: discovery, properties and potential for therapy. Cell Res 22, 288-304 (2012)). Specific examples of neural crest-derived tissue include, without limitation, dental pulp cells, dental pulp stem cells, and cells obtained from dental tissue (see, e.g., Sharpe PT. Dental mesenchymal stem cells. Development. 2016 Jul 1 ;143(13):2273-80), periodontal ligament tissue, apical papilla, and corneal stroma. Methods of identifying, isolating and preparing cells, including populations of cells comprising stem cells and induced stem cells, are broadly-known. Depending on the manner or use of the cells, autologous, allogeneic, or xenogeneic cells may be used to produce NTFs and/or cell structures according to in any aspect or embodiment of the methods described herein.

The cells used to prepare the nerve conduits, e.g., rolled and fused cell sheets, as described herein may be dental pulp cells. Dental pulp is the soft, connective tissue in the center of the tooth that contains fibroblasts, blood vessels, nerves and a population of stem/progenitor cells. These dental pulp stem/progenitor cells (DPCs) have been shown to be clonogenic and multipotent and can be easily obtained from autologous sources. One characteristic that makes DPCs unique in comparison to other commonly studied adult stem/progenitor cells, like those found in the bone marrow or adipose tissues, is that DPCs are embryonically derived from the cranial neural crest. Because of their developmental origins, DPCs have high capability for differentiating into other neural crest derived cells including neurons and neuron support cells. Furthermore, DPCs have been shown to express higher levels of NTFs when compared to bone marrow stromal cells and adipose-derived stem cells, likely due to their developmental origins. These characteristics make DPCs an ideal candidate for developing cellular therapies to deliver NTFs to damaged nerve tissues.

Dental pulp cells are typically isolated from extracted teeth, such as from third molars, supernumerary teeth, deciduous teeth, or teeth removed for orthodontic purposes. In aspects, pulp tissue is removed, and treated with a protease, such as a collagenase or dispase. Cells are separated from debris, e.g., by centrifugation or using a cell strainer, and are cultured in a culture dish in any suitable cell culture medium, as are broadly known, capable of supporting growth of dental pulp cells (hereinafter referred to as “dental pulp stem cell culture medium”). Culture medium useful for growth and expansion of mesenchymal stem cells can be used for growth of dental pulp stem cells. In one aspect, for example and without limitation, dental pulp culture medium consists of: 80% DMEM high glucose with Glutamax (Gibco), 20% fetal bovine serum (Atlanta Biologicals), and 100 U/ml penicillin and streptomycin (Gibco). Cell culturing medium is broadly-available from a multitude of sources. Stem cell culturing products are commercially available from a number of sources, including from Stemcell Technologies, Inc. of Cambridge, MA. In one aspect, ascorbic acid is added in order to strengthen the integrity of the formed sheet. In another aspect fibroblast growth factor 2 (FGF2) is added, for example, in the range of, for example, 1 ng to 50 ng per 2.5 mL of culture medium.

Cell sheets are a form of scaffold-free tissue engineering where cells proliferate to confluence and produce endogenous extracellular matrix to form a layer of tissue that can be separated from the substrate. Cell sheet technology presents a biomimetic and natural method for cell delivery since it does not involve any exogenous materials. Cell sheets have been studied for use in many regenerative applications including for myocardial, corneal, periodontal, and osseous repair.

A “neuron”, or alternatively a “nerve cell”, is a cell of the central nervous system, or peripheral nervous system of an animal, such as a human, that conducts nerve impulses. Neurons can be multipolar, bipolar, unipolar, or pseudounipolar, and include, without limitation, sensory (afferent) neurons, motor (efferent) neurons, association neurons, projection neurons, intrinsic neurons (interneurons), Purkinje cells, pyramidal cells, olifactory cells, retinal cells, and ganglion cells, among many others. Neural tissue is tissue that comprises one or more neurons. A “neurite” (also, “neuronal process”) is a projection from the cell body of a neuron, such as an axon or dendrite. Neurite outgrowth, characterized by neurite extension, is a characteristic of growth of neuronal development, and in the context of one aspect of the present disclosure, neuronal growth and repair.

A “cell growth matrix” is a mesh, matrix, particle, surface, hydrogel, porous structure, or other material upon which or into which a cell can be deposited and can be maintained in a living state, and often propagates (multiplies) in the presence of suitable cell growth media. A cell growth matrix can be manufactured from a single composition, or multiple compositions, such as synthetic and/or natural polymer compositions. A cell growth matrix may comprise cells and/or therapeutic agents. A “scaffold-free cell growth matrix” contains no synthetic polymeric compositions and is a natural product of cells and tissues. In the context of the present invention and disclosure, the described scaffold-free cell growth matrix is manufactured from artificially-manipulated cells to form an artificial, aligned extracellular matrix structure comprising an enriched population of cells that is rolled and fused into an artificial cylindrical structure that is useful as a nerve conduit for nerve repair.

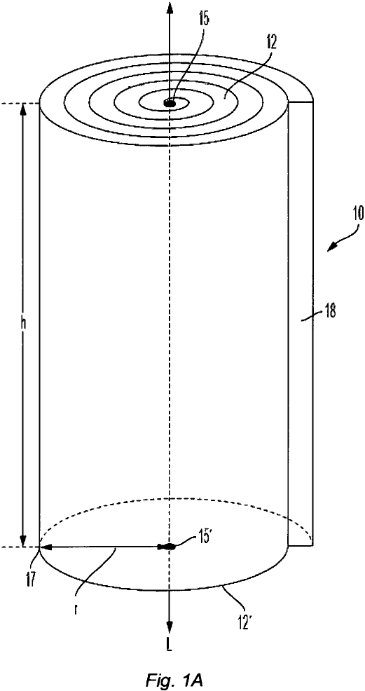

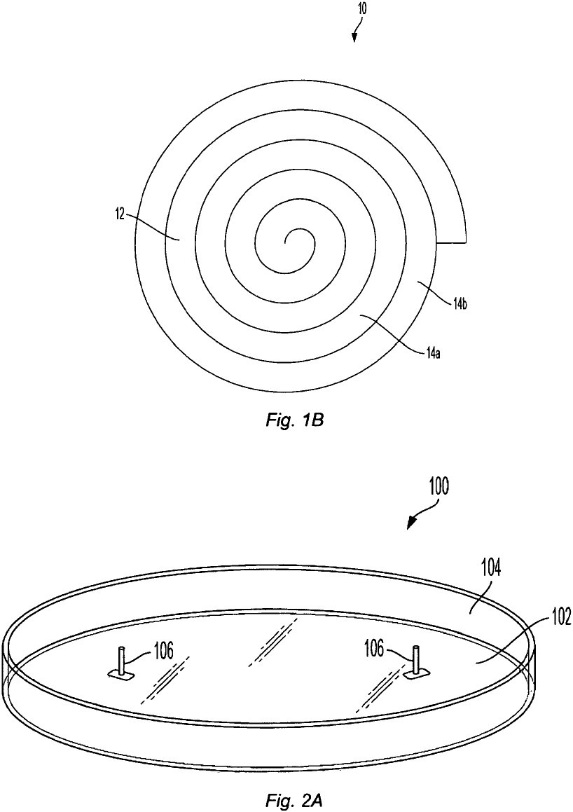

A cylinder is a solid geometric figure with straight parallel sides and a circular or oval, e.g., elliptical, cross section, having a height and one or more radii from a center point or one or more foci, and having a longitudinal axis extending perpendicular to the cross-sections. The term “cylindrical” refers to a three-dimensional geometric shape that is either geometrically perfect or generally having a shape of a cylinder, and may be generally cylindrical, cylindroidal, or similar shape. The rolled and fused cell sheet structures described herein are cylindrical, with the understanding that the nature of the described rolled sheet of cells and fused rolled sheet of cells will not necessarily be perfect cylinder or cylindroid, and may have an irregular circumference (e.g., not perfectly circular, elliptical, or ovoid) at any point along the longitudinal axis, and/or irregular radii taken at different points along the longitudinal axis. FIG. 1A is an exemplary, schematic diagram of a cylindrical roll 10 formed by rolling and fusing a cell sheet as described herein. The roll 10 has a first end 12 showing the spiral formed by the rolling of the cell sheet, with FIG. 1B showing an end-view of the first end 12 of the roll 10, depicting the adjacent layers 14a and 14b of the rolled cell sheet. Referring to FIG. 1A, the cylindrical roll 10 has a second end 12’, and center points 15 and 15’ are shown for the first end 12 and second end 12’, respectively. A longitudinal axis L of the roll 10 is depicted, along with a radius r extending as a line from the center point 12’ to the circumference 17 of the roll and perpendicular to the longitudinal axis L. The height h of the cylindrical roll 10, alternatively referred to as the length of the roll 10, is depicted, and is the distance between ends 12 and 12’, measured parallel to the longitudinal axis L. As shown, the radius r differs for any cross-section of the roll 10, depending on the direction from the center to the circumference because the structure is rolled from a sheet. For example a radius taken at an end 18 of the sheet may be larger than a radius taken at another point along the circumference of the sheet. As indicated above, the sheet of cells used to form the roll 10 may be circular or any shape, the cell sheet is expected to have a somewhat irregular thickness at different points, and culturing the roll to fuse the layers will likely affect the overall shape of the roll 10, therefore, the radius of the roll 10 measured at different points along the longitudinal axis and/or at different directions at any given point along the longitudinal axis L is expected to differ at those different points. As an example, the radius of a roll formed from a circular cell sheet will decrease at its ends. That structure may be used as-is for repair of a nerve, or strongly-tapered or irregular ends of the roll may be cut off prior to use to produce a cylindrical roll having flat ends.

A composition is “free” of a stated constituent if that constituent is not present in the composition or is present in insubstantial amounts that do not interfere, or that insignificantly interfere, with intended use and function of the composition.

A “population of cells” are cells refers to two or more cells. The cells in a population of cells may be the same, as in an enriched, purified, or clonally expanded population of stem cells. The cells in a population of cells may comprise different cell types, as in cells obtained directly from a tissue sample, comprising stem cells as well as differentiated cells. In the case of dental pulp, the initial cell population of cells may comprise: fibroblasts, odontoblasts, lymphocytes, macrophage, and stem cells (DPSCs), among other cells.

A nerve conduit in the form of a living cell structure provided that can be used as a NTF delivery system that can augment current facial nerve surgical treatment modalities to accelerate healing and improve functional recovery. These nerve conduits comprise only the cells and their extracellular matrix, and are minimally manipulated for forming the described nerve conduit. These nerve conduits, would supplement current surgical techniques by being placed at the site of a nerve injury or defect to induce axon outgrowth and the filling of gaps in nerves caused by nerve damage or defects. This technology is a simple and efficient manner to provide sustained NTF delivery in congruence with current surgical treatments. Through the delivery of NTFs and aligned cells and ECM components, the described nerve conduits would enhance axon regeneration compared to the current standard of care resulting in reduced regeneration time and improved functional outcomes.

Stem cells, such as neural crest-derived cell structures, such as dental pulp cell sheets meet all of the criteria of the ideal material for nerve repair. Ideal materials for nerve repair are biocompatible, are easily prepared and customizable to the size of the defect, and include bioactivity to promote axonal regeneration (Gaudin R, et al. Approaches to Peripheral Nerve Repair: Generations of Biomaterial Conduits Yielding to Replacing Autologous Nerve Grafts in Craniomaxillofacial Surgery. Biomed Res Int. 2016;2016:3856262. Epub 2016/08/25. doi: 10.1155/2016/3856262. PubMed PMID: 27556032; PMCID: PMC4983313). Cell structures, such as cell sheets, and rolled cell sheets as described herein, can be formed using autologous, allogeneic or xenogeneic cells, and they can be easily scaled up or down to fit defect size, and the cell structures are robust and can be easily handled by the surgeons for implantation. Furthermore, the cell structures express NTFs endogenously and improve axon growth. This technology addresses a current unmet clinical need to shorten regeneration time and improve functional outcomes for nerve, e.g., facial nerve, repair.

The scaffold-free nerve conduits, are robust and can easily be handled by surgeons for implantation. The DPCs in these nerve conduits express the genes for NTFs including brain-derived growth factor (BDNF), glial-cell derived neurotrophic factor (GDNF), and neurotrophin-3 (NT-3). Furthermore, the nerve conduits have the functional effect of promoting neurite formation and oriented extension in SH-SY5Y human-derived neuronal cells in vitro.

A neurotrophic factor is a compound or composition, e.g., a peptide or small protein, that regulates the proliferation, survival, migration, and/or differentiation of cells in the nervous system. Non-limiting examples of neurotrophic factors include: nerve growth factor (NGF), ciliary neurotrophic factor (CNTF), neurotrophin-3 (NT-3), neurotrophin-4, neurotrophin-5, glial-cell derived neurotrophic factor (GDNF), pleiotrophin protein (neurite growth-promoting factor 1), midkine protein (neurite growth-promoting factor 2), and brain-derived neurotrophic factor (BDNF).

A method of producing neurite outgrowth in a neuron is provided. The method may be performed in vitro or in vivo. The method comprises, first, culturing a population of cells comprising stem cells, such as neural crest-derived cells, e.g., dental pulp cells, to produce a sheet or other shape. The sheet is rolled into a cylindrical shape and is cultured to fuse the roll into a cylinder, forming a nerve conduit. The cells and ECM are aligned in a direction substantially parallel to the longitudinal axis of the cylindrical nerve conduit, either by producing the cell sheet on a linearly-grooved substrate, or anchoring ends of the rolled sheet during culture to fuse the rolled sheet into a cylinder. The living nerve conduit is implanted between ends or stubs of a damaged nerve, and induces neurite outgrowth from the ends of the damaged neuron, thereby repairing the nerve.

Nerve damage can be from trauma, disease, or surgical injury, and can be a peripheral nerve, or CNS nerve tissue, such as spinal cord tissue. The method comprises implanting a living nerve conduit as described herein, comprising a rolled and fused sheet of cultured cells comprising stem cells, e.g., dental pulp cells, or other neurotrophic factor-producing cells, e.g., stem cells, and/or progenitor cells isolated from neural crest-derived tissue (“neural crest-derived cell”), at the site of a nerve injury in patient. Although autologous cells may be preferred in instances, allogeneic or xenogeneic cells may be used. In one aspect, the structure, e.g., sheet, is formed by ex vivo culturing of the cells, e.g., cell populations comprising stem cells or neural crest-derived cells, such as a population of cells derived from the neural crest and comprising, e.g., progenitor and stem cells, for example and without limitation, dental pulp cells, typically to confluence or post-confluence, in order to produce a sufficiently robust extracellular matrix ex vivo. The nerve injury may be, for example and without limitation, a facial nerve injury, an injury to any peripheral nerve, or a spinal cord injury. The method also optionally comprises obtaining cell populations comprising stem cells or neural crest-derived cells, such as a population of cells derived from the neural crest and comprising, e.g., progenitor and stem cells, for example and without limitation, dental pulp cells, e.g., from the patient, and culturing the cells to confluence or to post-confluence.

To form a solid tissue structure, such as a cell sheet, cells are grown under suitable conditions, for example, past confluence, until a sheet or other solid structure is formed having sufficient mechanical strength for handling. The cells plus the extracellular matrix produced by the cells form a living structure, such as a living sheet (“cell sheet”), and rolled and fused nerve conduit that can be removed from the culture dish, physically manipulated, and implanted into a patient adjacent to a nerve, e.g., by suturing or gluing, e.g., with fibrin glue, as is broadly-known in the medical arts. Any suitable tissue culture dish or surface can be utilized to produce the cell structures or sheets, such as petri dishes, multi-well plates (e.g., a 6-well tissue culture plate as used below in the Example), flasks, or other surfaces. Cell sheets can be cultured on microscope slides within a tissue culture flask or any other suitable surface. Surfaces of different stiffness or elasticity can be used to grow the cell sheet. In aspects, the surface on which the cell sheet is grown or produced can be coated with a cell adhesion composition, such as laminin or fibronectin, as are known in the cell-culturing arts. As described herein, the surface on which the cell sheet is produced may comprise linear grooves.

Prior to implantation in a patient to treat a nerve injury or defect, the cell structure or sheet may be washed to remove cell culture medium components, such as xenogeneic serum, antibiotics, and any other undesirable constituents. The cell sheet may be washed in any suitable solution, such as serum-free medium, phosphate-buffered saline, or normal (0.9 % w/v) saline, optionally including suitable antibiotics.

As indicated above, provided herein are methods of making nerve conduits that find use in nerve regeneration. Also provided herein are nerve conduit devices useful in nerve repair or regeneration. Further, methods of treating patients with nerve damage or defects are provided. The method comprising growing a stem cell-containing population of cells on a substrate to confluence produce a cell sheet. The cell sheet comprises live cells and deposited extracellular matrix. The cell sheet is removed from the surface it was formed on, by any useful method, typically mechanical. The cell sheet is rolled into a cylindrical shape and is subsequently cultured in the rolled shape to fuse the cylinder into the cylindrical shape by interaction, e.g., adhesion of cells of adjacent layers of the rolled structure. The cells may be any cell population as described above, comprising totipotent, pluripotent, or multipotent stem cells, mesenchymal stem cells, neural crest-derived cells, or dental pulp stem cells. The cell sheet may be treated with suitable factors to induce production of neurotrophic factors, or to induce differentiation of the cells to a neurotrophic factor-producing cell type, such as Schwann cells, for example as described in Example 4.

A Schwann cell is a specialized type of glial cell of the peripheral nervous system that plays a role in the development, maintenance, function, and regeneration of peripheral nerves, including production of neurotrophic factors. Different types of Schwann cells (a Schwann cell-like phenotype) may be produced from stem cell precursors, such as non-myelinating (e.g., Remak), myelinating, and repair Schwann cells (See, e.g., Jessen KR, et al., Schwann Cell Precursors; Multipotent Glial Cells in Embryonic Nerves. Front Mol Neurosci. 2019;12:69 and Sakaue M, et al., Human epidermal neural crest stem cells as a source of Schwann cells. Development. 2015;142(18):3188-3197).

The cells may be cultured on a substrate having grooves, e.g., linear grooves with gaps between the grooves in the micron range, e.g., from 100 nm to 25 µ (microns) between adjacent walls of adjacent grooves. The gap between grooves may be readily ascertained in the context of square-profile grooves as shown in Example 1, below, but may be an average distance between adjacent grooves, or a distance between portions of the facing walls of adjacent grooves that have a rounded profile or that have walls extending from the base substrate that are not perpendicular to the substrate, e.g., slanted or sloped. Grooves can have, without limitation, a square, trapezoidal, triangular, saw-tooth, or rounded profile. The result of culturing on a grooved substrate is that cells and ECM of the cell sheet align in the direction of the grooves. The cell sheet is then rolled so that the direction of alignment of the cells is in the direction of the longitudinal axis of the resultant cylindrical roll. The rolled cell sheet is then cultured to fuse the cell sheet into a cylindrical structure, forming the cylindrical nerve guide.

The grooved substrate or a flat substrate, may be manufactured from any materials that is supportive of cell-growth and formation of a cell sheet as described. The grooved substrate may be manufactured from a polymeric composition, and in one example, the polymeric composition comprises a polysiloxane, or polydimethylsiloxane, as in SYLGARD® products. SYLGARD® 184 is used in the examples below, but other polysiloxane, e.g. polydimethyl siloxane compositions may be utilized. To manufacture the substrate, e.g. a grooved substrate, the polymeric composition is formed or molded on a suitable template. Where the substrate is flat, the template can be any compatible surface, such as, for example and without limitation, a silanized glass surface. To produce a grooved substrate, a silicon wafer may be modified, e.g., grooved, using standard lithography methods as are broadly-known in the fabrication arts. The silicon wafer may be silanized and can serve as a suitable template for production of the grooved substrates for production of an aligned cell sheet. The grooves may be linear and/or parallel, and may be regularly-spaced, e.g., with gaps (in the case of the cell-growth substrate) or ridges (in the case of the silicon template) of less than 250 microns (µ), less than 100 µ, less than 50 µ, less than 25 µ, or less than 10 µ in width, such as ranging from 1 µ to 10 µ in width.

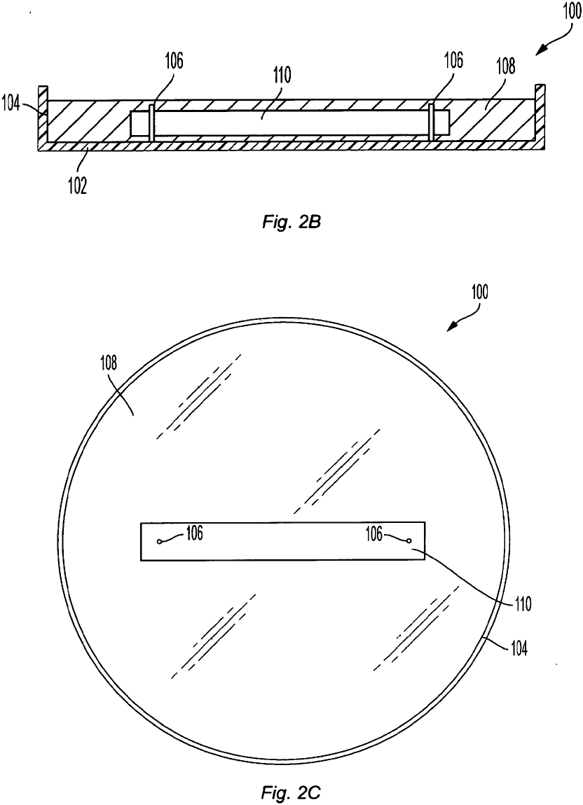

Once removed from a substrate and rolled, a cell sheet tends to contract. In an alternative method to aligning the cells on a grooved substrate, the cells, as described above, may be cultured on a flat surface as is common in tissue culture, separated from the surface, and is rolled into a cylindrical shape. The rolled sheet is then cultured to fuse adjacent layers while having its ends anchored. Stresses on the anchored cylinder aligns the ECM and cells longitudinally essentially as described above for the cell sheet grown on a grooved substrate. The rolled cell sheet may be anchored using pins in a tissue culture vessel. FIGS. 2A - 2C depict an example of this anchoring technique. FIG. 2A shows a culture vessel 100 having a bottom 102 and walls 104. A lid (not shown) may be used to cover the vessel 100. Pins 106 are depicted, which are used to anchor the cell sheet roll. A cross-sectional view of the culture vessel 100 of FIG. 2A in use is shown in FIG. 2B, depicting the bottom 102, walls 104, and pins 106 of the vessel 100, and further showing cell culture medium 108, and a rolled, cylindrical cell sheet 110 anchored by the pins 106, which penetrate through the cylindrical, rolled cell sheet 110. FIG. 2C provides an overhead view of the vessel 100 depicted in FIG. 2B, depicting the walls 104, the pins 106, the medium 108, and the cylindrical, rolled cell sheet 110. The vessel and configuration of FIGS. 2A-2C are merely exemplary, and the vessel and pins, or more generally anchors, may have any useful shape, size, and configuration, e.g., flasks or bioreactors as are broadly available and understood in the cell and tissue culture arts. Suitable pins or other anchors may be affixed to a surface of a standard tissue culture vessel, or may be formed as an integral part of the vessel. The methods described herein may be partially or fully automated using suitable computer-implemented robotic and fluidic mechanisms and methods. Cell culture conditions, medium, devices, etc. may be varied so long as they permit cell growth and formation of nerve conduits as described herein.

In an alternative version of the method and construct shown in FIGS. 2A-2C, the cell sheet is not rolled, but is anchored, e.g., using two pins. While rolling, or allowing the sheet to form a roll on detachment to the substrate may be useful, the rolling is not always necessary or desirable. The cell sheet may be anchored and subsequently cultured, and due to stresses placed on the cell sheet from culturing while anchored, the cells will align, forming an aligned construct useful, e.g., in nerve regeneration.

Cells may be obtained from any suitable source, e.g., from a patient, or from banked cells or tissue of a patient, and a nerve conduit may be formed as described herein. The nerve conduit may be cryopreserved according to any useful protocol, as are broadly-known. The cryopreserved nerve conduit may be stored in a frozen state, e.g., at -20° C. or colder. The frozen cryopreserved nerve conduit may be shipped in a frozen state, e.g., with cold packs or dry ice. After cryopreservation and storage, the nerve conduit is thawed and cultured under suitable conditions to preserve viability of the nerve conduit. Once the cryopreserved nerve conduit is restored in culture, it may be used to repair a nerve of a patient. The cells may be autologous to the patient. The cryopreserved nerve guide may be stored in a frozen state in any suitable container, such as, without limitation, a vial or foil package, and can be shipped in the same packaging, with dry ice or a suitable cold-pack.

Additional therapeutic agents may be implanted or administered locally with the nerve conduit, such as any useful cytokine or chemoattractant can be mixed into, mixed with, or otherwise combined with the nerve conduit as described herein. For example and without limitation, useful components include growth factors, interferons, interleukins, chemokines, monokines, hormones, and angiogenic factors. In certain non-limiting aspects, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF),corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minnesota; Biovision, Inc, Mountain View, California; ProSpec-Tany TechnoGene Ltd., Rehovot, Israel; and Cell Sciences®, Canton, Massachusetts. Other drugs that may promote wound healing and/or tissue regeneration may also be included.

Although the examples below describe use of a rolled and fused living nerve conduit in repair of facial nerves, the nerve conduits are useful for repair of any peripheral nerves, by implantation of the nerve conduit at the site of a gap in a nerve due to a nerve injury or defect, and the nerve conduit may be anastamosed between ends (stumps) of a damaged nerve. Typically, the nerve conduit is prepared from a patient’s own tissue and cells, such as from dental pulp from an extracted tooth, and therefore is autologous, though allogeneic or xenogeneic tissue may be used.

### EXAMPLES

The goal of this study is to improve the current standard of care for treating nerve injuries, such as facial nerve injuries. Nerve injuries or defects, such as facial nerve injuries or defects may result from trauma, tumor ablation, and iatrogenic surgical injury. The current gold standard for peripheral nerve repair is to replace damaged tissue with an autograft, however cable grafting comes with slow or incomplete recovery of volitional movement. One factor contributing to these results is diminished Schwann cell support. Schwann cells provide neurotrophic factors (NTFs), which are known to promote neuron survival and axon extension. The optimal peripheral nerve conduit will supply both these trophic cues to stimulate axon growth and also a suitable surface that provides directional guidance to promote axon extension enabling the axons to bridge the nerve defect. The dental pulp contains a population of stem/progenitor cells that are being investigated for several clinical applications. These dental pulp cells (DPCs) have been shown to endogenously express high levels of NTFs, a characteristic likely due to their neural crest origins. Cell sheets are a form of scaffold-free tissue engineering where cells proliferate to confluence and produce endogenous extracellular matrix to form a layer of tissue that can be separated from the substrate. Our preliminary data shows that scaffold-free DPC sheets are effective NTF delivery systems expressing sufficient NTFs to induce neurite extension in vitro and enhance the regeneration when wrapped around facial nerve defects models in vivo. In this study we plan to enhance the design of our scaffold-free DPC sheets so that in addition to providing NTFs, these engineered tissues will also provide guidance cues that will promote directional axon extension.

The overall goal of these examples is to develop a scaffold-free nerve conduit, engineered using stem cell-containing populations of cells, such as DPCs or other neural crest-derived tissue, and their aligned ECM, to promote nerve regeneration, e.g., facial nerve regeneration. DPC conduits with linearly aligned ECM will provide (1) a continual supply of NTFs and (2) a substrate that provides guidance cues to direct axon extension. The cells used in Examples 1-6 may be described as DPCs, but any suitable stem cell-containing population of cells, or a purified or enriched population of stem cells, e.g. mesenchymal stem cells, or cells or stem cells obtained from neural crest-derived tissue may be used. The cells may be commercially-obtained or may be banked (cryopreserved) tissue, such as banked dental pulp, adipose, or bone marrow tissue, and may be autologous, syngeneic, or allogeneic.

Preliminary data demonstrated that DPCs form manipulatable NTF-secreting cell sheets capable of inducing axon extension (See, International Patent Publication No. WO 2019/169234, incorporated herein by reference in its entirety). When cultured on a smooth substrate, DPCs formed a robust cell sheet that could be easily detached from the culture dish and handled. The DPCs in the cell sheets express the NTF genes as detected by quantitative real-time polymerase chain reaction (qPCR) and proteins for BDNF, GDNF, and NT-3 as assessed by enzyme-linked immunosorbent assay (ELISA).

The following materials and methods apply to the examples below (See, International Patent Publication No. WO 2019/169234).

Dental pulp cells isolation and engineering of scaffold-free cell sheets: Dental pulp was isolated from adult human 3rd molars, the teeth were free from carious lesion or other oral infections and they were collected within 24 hours of extraction and transported to lab in phosphate buffered saline with penicillin and streptomycin. A digestion cocktail containing collagenase (3 mg/ml) and dispase (4 mg/ml) (EMD Millipore Corporation and Worthington biochemical, USA) was used to digest dental pulp and obtain a total population of dental pulp cells. The isolated cells were expanded and cultured in growth medium (GM) made up of Dulbecco’s Modified Eagle Medium (Gibco Life technologies corporation, USA), 20% fetal bovine serum (Atlanta biological, USA) and 1% penicillin and streptomycin (Gibco Life technologies corporation, USA). Further, upon reaching 80% confluence the cells were passaged and cryogenically stored in liquid nitrogen at -196° C. for future experiments. The multipotency of the isolated DPC was verified by inducing differentiation towards osteogenic lineage; the deposition of mineralized matrix was confirmed through Alizarin red staining.

Dental pulp cells from passage 2-4 were used to engineer cell sheets: DPC were plated onto 6 well plate at an initial seeding density of 200,000-400,000 cells per well in growth medium supplemented with 50 µg/ml L-Ascorbic acid (Sigma-Aldrich, USA), and 5 ng/ml Fibroblastic Growth Factor 2 (Peprotech, USA). The dental pulp cells were cultured with medium change once every 2-3 days for 10-12 days to form robust cell sheet that could be easily handled with forceps. Although DPCs from passage 2-4 were used, technically DPCs from different passage numbers may be used.

Quantification of cell number in engineered DPC sheets: Scaffold-free engineered DPC sheets were rinsed with PBS twice followed by their digestion by incubating in 350 µl of TrypLE express (Gibco Life technologies corporation, USA) at 37° C. for 5-7 minutes. The detached cells were suspended in 1 ml of growth media and they were counted in hemocytometer with trypan blue stain. Cell viability of the dental pulp cells was verified with trypan blue stain; the dead cells absorbing trypan blue stain were excluded from the count.

Histological characterization of engineered DPC sheets: Engineered DPC sheets were washed with PBS twice and fixed with 10% formalin for 20 minutes and stored in 70% ethanol at 4° C. overnight. The cell sheets were processed in tissue processor (LEICA ASP300S, Leica Instruments GmbH Germany) for standard paraffin embedding. Processed cell sheets were sectioned at 5 microns thickness in microtome (LEICA RM2135 style, Leica Instruments GmbH Germany) and incubated at 60° C. for 15 minutes. The cell sheet sections were processed for Hematoxylin and Eosin (H&E) and immunostaining by deparaffinizing and rehydrating the sectioned samples by series of washes in xylene, ethanol and water.

Hematoxylin and Eosin stain: The hydrated samples were stained with H&E (Richard-Allan scientific) by a series of washes in hematoxylin, water, NU-CLEAR™ (acid/alcohol), bluing, water, ethanol, eosin, ethanol, xylene and mounted with xylene based mounting media (Thermo scientific, USA). The images were captured using ZEISS Scope.A1 AXIO microscope.

Immunostaining: Chemical and immunofluorescent staining was performed on fixed cell sheets or histological sections of cell sheets. Engineered DPC sheets were washed with PBS twice and fixed with 10% formalin for 20 minutes. Cell sheets were stained with phalloidin and 4’,6-diamidino-2-phenylindole DAPI to visualize cellular cytoskeletal organization and nuclei, respectively. Cell sheets were also immunostained using a type I collagen anti-body to visualize collagen organization. For analyses on histological sections, the hydrated sample slides were incubated in 10 mM citrate + 0.05% triton X at 60° C. overnight for heat induced epitope retrieval. Further, sample sections were permeabilized with 0.1% triton X and incubated with 5% goat serum for 1 hour. Sections were incubated with either type 1 collagen antibody (Anti-collagen | antibody ab34710, Abcam) at 1:200 concentration overnight at 4° C. or in blocking solution as negative control. The sections were stained with secondary antibody Alexa Fluor 488 (ThermoFisher, USA) at 1:500 concentration for one hour followed by counterstaining with 4’,6-diamidino-2-phenylindole DAPI (Sigma-Aldrich, USA) stain for nuclei at 1:500 concentration. The sections were mounted with aqueous mounting media (Shandon Immu-mount USA). The images were captured using Nikon ECLIPSE Ti microscope and processed in imageJ software.

Analysis of neurotrophic factor gene expression using reverse transcription polymerase chain reaction (RT-PCR): Scaffold-free engineered DPC sheets were harvested after 10-12 days in culture and RNA was extracted using QIAGEN RNEASY® Mini Kit following the manufacturer’s protocol. Briefly, the DPC sheet was lysed and homogenized using RLT buffer and mixed with 1 volume of 70% ethanol. This was followed with a series of washes and centrifugation with RW1 and RPE buffer before collecting RNA in 30 µl of RNase-free water. The quantity and quality of the RNA was measured using nanophotometer (NANODROP™ One Thermo Fisher, USA). RNA was collected from cell sheets cultured in media containing +/- FGF2. RNA was also isolated from DPC at the time of plating and human embryonic kidney fibroblasts (hek 293t) as control samples.

RT-PCR was performed with TAQMAN™ PCR kit (Applied Biosystems) to analyze the expression of neurotrophic factor genes using primers for human BDNF, GDNF and NT3 (TAQMAN™ Gene Expression Assays) and GAPDH was used as housekeeping gene. The assay was conducted in QUANTSTUDIO™ 6 Flex (Applied Biosystems Life technologies). The obtained data was analyzed and Ct values greater than 35 were considered as negative readings, the fold change for each of the sample was calculated using the ΔΔCt method. First, the ΔCt value was calculated by normalizing the Ct value of each sample gene to that of the housekeeping gene. Then, the difference in the ΔCt values between the experimental and control group was calculated as the (ΔΔCt). The fold change for each sample was calculated as 2(-ΔΔCt).

Detection of neurotrophic factor protein secretion by DPC sheets using enzyme linked immunosorbent assay (ELISA): Conditioned medium (CM) was obtained from DPC sheets cultured in growth medium, the CM was spun down at 2000 rpm for 5 minutes to remove any cellular debris and stored at -80° C. for future experiments. The uncultured growth media was aliquoted and incubated at 37° C. for 48 hours and stored at -80° C. for future use as control samples. The amount of BDNF, GDNF and NT3 proteins secreted by DPC in the cell sheet was measured by ELISA using Human BDNF PICOKINE™ ELISA Kit EK0307, Human GDNF PICOKINE™ ELISA Kit EK0362 (Boster Biological technology CA, USA) and Human NT-3 ELISA Kit (RayBiotech, USA) respectively. The ELISA assay was performed following manufacturer’s protocol, briefly conditioned medium, growth medium and reconstituted proteins were added to the antibody pre coated 96-well plates and incubated at 37° C. for 90 minutes followed by incubation with biotinylated antibody at 37° C. for 60 minutes. The plates were washed with 0.01 M PBS for three times and then incubated with Avidin-Biotin-Peroxidase Complex (ABC) at 37° C. for 30 minutes. The plate was washed with 0.01 M PBS for five times and incubated with TMB (3,3’,5,5’-tetramethylbenzidine) solution in dark at 37° C. for 20-25 minutes which developed blue color. Further TMB stop solution was added which changed the color to yellow immediately and the plate was read at optical density (O.D) absorbance value of 450 nm in a spectrophotometer (SYNERGY™ H1 microplate reader, BIOTEK USA) within 30 minutes of adding the TMB stop solution. The quantity of secreted proteins (pg/ml) were calculated against standard curves produced using recombinant protein concentrations provided by the manufacturer using linear regression. The experiment was repeated 3 times using cell sheet formed from cells isolated from 3 different individuals. Mean value ± standard deviation represent the biological triplicate; average across 3 experiments.

In vitro neurite outgrowth assay using SH-SY5Y neuroblastoma cells: To assess the functional effect of the neurotrophic factors secreted by DPC and the aligned ECM comprising the cell sheets, cell sheets were co-cultured with SH-SY5Y neuroblastoma cells (ATCC CRL-2266). SH-SY5Y neuronal cells neuronally pre-differentiated with culture media containing DMEM/F12 with 10% fetal bovine serum for 24 hours followed by neuronal induction with 10 uM retinoic acid (ACROS organics, USA) for 48-72 hours of incubation. The neuronally pre-differentiated SH-SY5Y neuroblastoma cells were then plating onto of the DPC sheets, and were further cultured for 3-6 days with media change once every 2-3 days. After treatment the cells were washed with PBS twice and fixed for 20 minutes with 4% paraformaldehyde (Sigma-Aldrich, USA) prepared fresh. The SH-SY5Y cells were washed with PBS twice and stained for immunostaining with anti-tubulin βIII antibody (Biolegend, USA) and DAPI at concentrations of 1:250. The images were captured with Nikon ECLIPSE Ti microscope.

Neurite extensions of SH-SY5Y neurons were manually quantified with imageJ software where neurite length and direction of extension were measured.

Statistical Analysis: The data is presented as means ± standard deviations. qRT-PCR data was analyzed by comparing the fold change results between treatment and control group using one-way ANOVA with post-hoc Tukey test. ELISA data was analyzed using the final picogram concentrations between treatment and control group by one-way ANOVA with post-hoc Tukey correction. All the statistical tests were done in Graphpad Prism software and statistical difference at p value less than 0.05 was considered significant.

### Example 1 - Evaluation of Patterned Growth Substrates

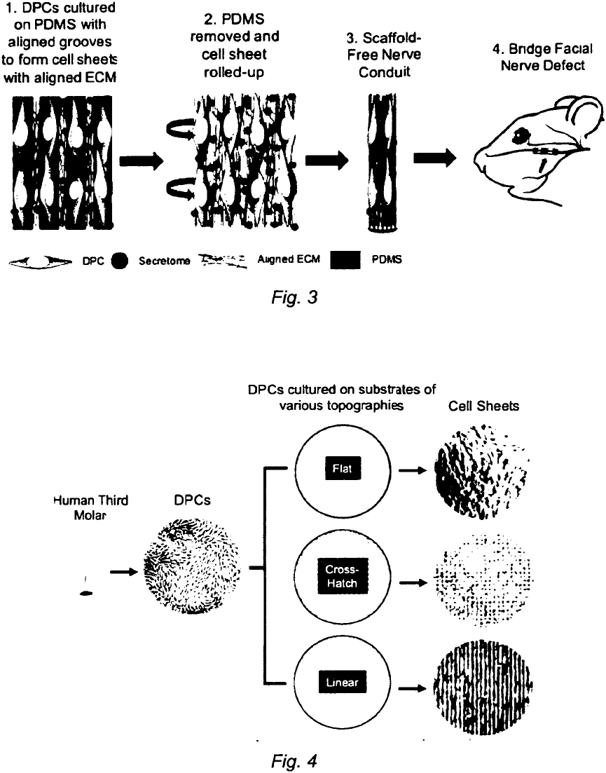

FIG. 3 provides a schematic outline of the method of preparing and employing an exemplary nerve conduit as described in Examples 1, and 2, as applied to a rodent experimental model. The same workflow and therapeutic end-use can be employed in nerve repair at any suitable location of nerve damage or defect, and can be employed in any animal, or mammal in a medical or veterinary setting. Non-limiting examples of suitable therapeutic subjects include humans, non-human primates, dogs, cats, horses, sheep, cows, pigs, reptiles, and birds.

As illustrated in FIG. 4, dental pulp cells were extracted from human third molars and were cultured on PDMS substrates. Dental pulp cells were isolated from human third molars and expanded in dental pulp stem cell culture medium. Dental pulp was isolated from adult human 3rd molars, the teeth were free from carious lesion or other oral infections and they were collected within 24 hours of extraction and transported to lab in phosphate buffered saline with penicillin and streptomycin. A digestion cocktail containing collagenase (3 mg/ml) and dispase (4 mg/ml) (EMD Millipore Corporation and Worthington biochemical, USA) was used to digest dental pulp and obtain a total population of dental pulp cells. The isolated cells were expanded and cultured in growth medium (GM) made up of Dulbecco’s Modified Eagle Medium (Gibco Life technologies corporation, USA), 20% fetal bovine serum (Atlanta biological, USA) and 1% penicillin and streptomycin (Gibco Life technologies corporation, USA). Further, upon reaching 80% confluence the cells were passaged and cryogenically stored in liquid nitrogen at -196° C. for future experiments. The multipotency of the isolated DPC was verified by inducing differentiation towards osteogenic lineage; the deposition of mineralized matrix was confirmed through Alizarin red staining.

For the present examples, the DPCs were plated onto a 6-well tissue culture plate at an initial cell density of approximately 20,000 cell/cm². The cells were cultured in a medium containing DMEM high glucose with Glutamax, 20% fetal bovine serum, 100 U/ml penicillin and streptomycin, 50 mg/ml ascorbic acid, and +/- 20 ng fibroblast growth factor 2 (FGF2). DPCs formed robust sheets within 10 days of culture. These conditions facilitate the DPCs to produce sufficient extracellular matrix to form a robust cell sheet.

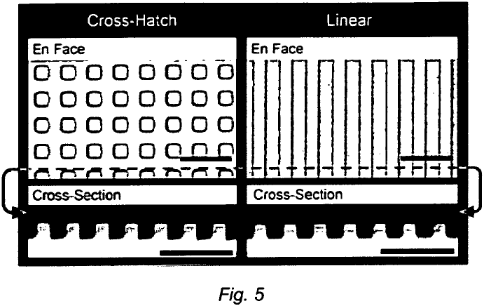

Substrates comprising an array of parallel grooves 10 µm wide, spaced 10 µm apart, and 5 µm deep, or cross-hatched features of similar width and depth, were generated by curing polydimethylsiloxane in molds with the negative features; flat PDMS substrates were used as controls. Dimensions were verified by light microscopy. In brief, the microgrooved substrates were designed in AutoCAD. Molds were fabricated on silicon wafers using standard lithography methods and subsequently coated with silane. Two types of molds were generated to generate substrates that have either: 1) an array of parallel lines 10 µm wide, spaced 10 µm apart, and 5 µm deep, or 2) a cross hatch of grooves spaced 10 µm wide and spaced 10 µm apart oriented 90 degrees relative to each other and 5 µm deep. Sylgard 184 polydimethylsiloxane (PDMS;Dow Corning, Midland, MI, USA) was mixed at a 10:1 base to curing agent ratio and cast into the molds to form 1.3 x 1.5 cm substrates. Dimensions were verified by light microscopy using a Nikon TE2000U microscope (FIG. 5). To promote cell adhesion, as needed, PDMS surfaces may be coated with laminin, rinsed and UV sterilized.

Cell alignment in the cell sheets was evaluated based on nuclei alignment. RT-PCR and ELISA were used to measure gene and protein expression of NTFs: BDNF, GDNF, and NT-3.

DPCs and neural cells were co-cultured on the produced sheets (flat, cross-hatched, and linear substrate), and neurite expression and alignment direction were ascertained. Briefly, to promote cell adhesion, PDMS surfaces were fitted into wells of 6-well plates and coated with laminin. DPCs were plated onto the substrates at an initial density of 4x10⁵ cells/well in a GM, and cell sheets were formed within 10 days. The cells become over-confluent and embed in the ECM they produce.

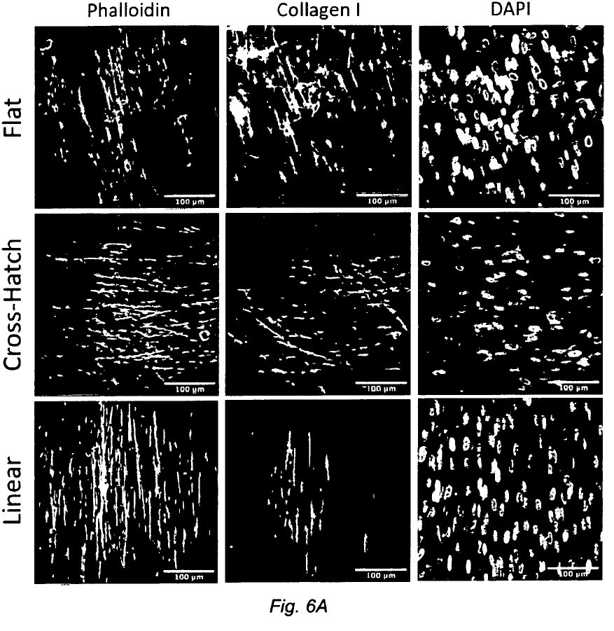

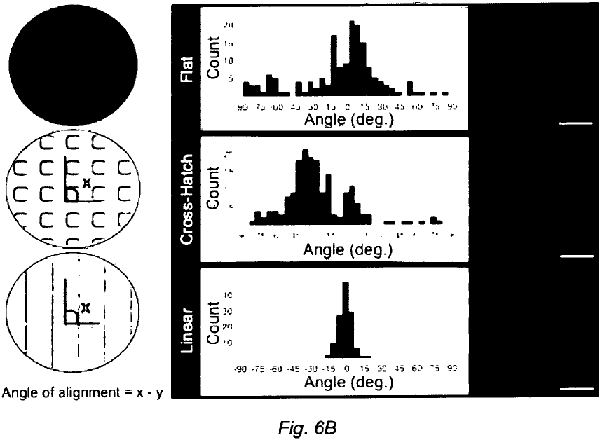

Linear grooved substrates induced DPCs to align and produce aligned, collagenous ECM. Phase contrast images show that DPCs align in parallel when cultured on substrates with aligned microgrooves, and this is further substantiated by phalloidin staining, which allows visualization of the cytoskeleton (FIG. 6A). This cellular organization is lacking in DPC sheets engineered on flat or cross-hatched substrates. Furthermore, DPC sheets cultured on parallel microgrooves produce an aligned collagenous ECM, as detected by immunostaining against type I collagen (FIG. 6A). Quantification of nuclear alignment showed that DPC nuclei have increased alignment relative to DPCs cultured on the flat or cross-hatched substrates (FIG. 6B). This data substantiates that DPCs have increased cellular and ECM alignment when cultured on linearly grooved substrates.

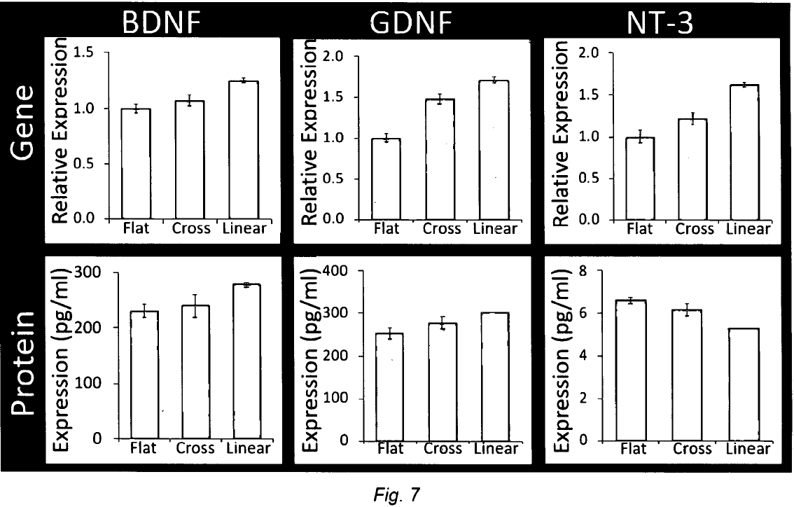

NTF gene expression is depicted in FIG. 7. Linearly-aligned DPCs expressed similar levels of NTFs than un-aligned DPCs. Therefore, alignment of DPCs had no significant effect on NTF expression.

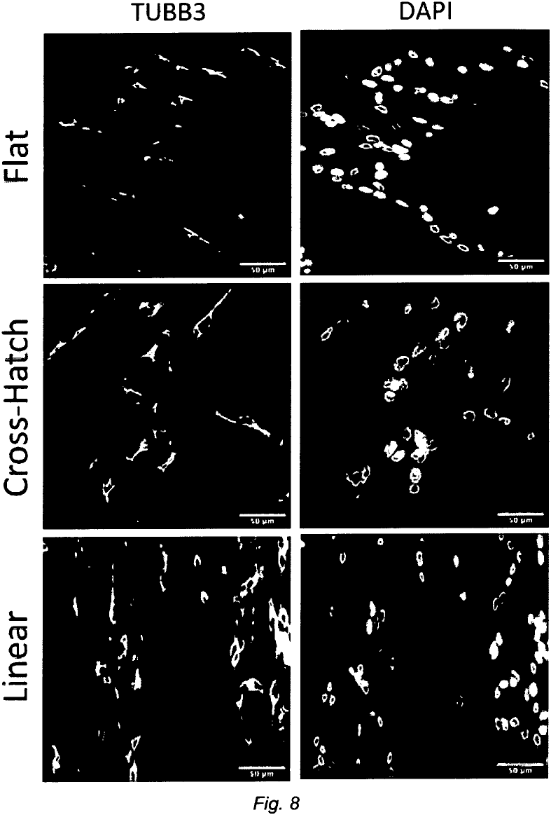

Neuronally pre-differentiated SH-SY5Y neuroblastoma cells extend neurites that are oriented in the direction of the aligned ECM when cultured on DPC sheets formed on a grooved substrate. We plated neuronally pre-differentiated SH-SY5Y cells directly on top of the cell sheets. These neuronal cells formed neurites on cell sheets generated on flat, cross-hatched, and linearly grooved substrates. Neurite outgrowth was oriented parallel to the aligned ECM of DPC sheets generated on the grooved substrate, whereas neurites were not oriented in any predictable direction when the neuronal cells were cultured on DPC sheets formed on flat or cross-hatched substrates. This data indicates that the aligned ECM effectively provides guidance cues to direct axonal growth. Cells were stained with Dil ((2Z)-2-[(E)-3-(3,3-dimethyl-1-octadecylindol-1-ium-2-yl)prop-2-enylidene]-3,3-dimethyl-1-octadecylindole; perchlorate), TUBB3 (Tubulin Beta 3 Class III), and DAPI. FIG. 8 shows the staining, and only neural cells grown on the linear substrate-grown DSCs aligned.

### Example 2 - Formation of a Cylindrical Nerve Guide

DPCs were cultured on a grooved substrate essentially as described in Example 1 to form a confluent sheet of cells. As above the cell culture medium was DMEM with 20% fetal bovine serum, 50 µg/ml L-Ascorbic acid, 5 ng/ml fibroblast growth factor, 1% penicillin-streptomycin. The sheet of cells was separated from the grooved PDMS substrate and rolled into a cylinder. The rolled cylinder of cells was then cultured under the same conditions and in the same growth medium overnight, e.g. for eight to 24 hours, in which time, the rolled sheet fused due to adhesion and migration of cells between adjacent layers of the rolled sheet.

### Example 3 - Alternative Method of Forming a Cylindrical Nerve Guide

DPCs were cultured on a flat substrate such as a PDMS substrate, or a cell culture vessel surface, essentially as described in Example 1, to form an over-confluent sheet of cells. As above the cell culture medium can be DMEM with 20% fetal bovine serum, 50 µg/ml L-Ascorbic acid, 5 ng/ml fibroblast growth factor, 1% penicillin-streptomycin. The sheet of cells are separated from the substrate and are either rolled into a cylinder or allowed to naturally contract. The rolled cylinder or contracted sheet of cells is anchored at two points, e.g., each of its ends in the case of a rolled sheet, using pins attached to a surface of a culture vessel and is cultured to form a fused, cylindrical nerve guide as described in Example 2, for instance overnight, e.g., from eight to 24 hours. Tension on the rolled cell sheet can cause cells and ECM between the pins to align, essentially as described with respect to the aligned cells depicted in Example 1. Where the sheet is not rolled, the cells in the sheet are expected to align, forming a suitable nerve guide. The two pins may be placed in the rolled sheet approximately 7 mm apart. Once the cells are aligned, and in the case of a rolled sheet, the cylinder of cells is fused, the construct can be used as a nerve conduit.

### Example 4 - Differentiation of DPCs to Schwann Cells

The DPCs or any stem cell-containing population, such as neural crest tissue or stem cells obtained from neural crest tissue, used in any one of Examples 1-3 may be differentiated to a Schwann cell-like phenotype. To induce Schwann cell differentiation, once the DPCs reach confluence in GM, they are subjected to Schwann cell differentiation protocol. For example, first, the DPCs are cultured in serum free medium containing 1 mM β-mercaptoethanol (BME) for 24 h followed by 72 h of culture in basal medium containing 35 ng/ml all-trans-retinoic acid. The medium is then switched to Schwann cell differentiation media (SCDM) comprising GM supplemented with 5 µM forskolin, 10 ng/ml fibroblast growth factor 2, 5 ng/ml platelet-derived growth factor-AA and 200 ng/ml heregulin-β-1. This protocol has previously been shown to induce DPC, BMSC, and ASC differentiation towards a Schwann cell-like phenotype (see, e.g., Martens W, et al., Human dental pulp stem cells can differentiate into Schwann cells and promote and guide neurite outgrowth in an aligned tissue-engineered collagen construct in vitro. Faseb J. 2014;28(4):1634-43; Sanen K, et al., Engineered neural tissue with Schwann cell differentiated human dental pulp stem cells: potential for peripheral nerve repair? J Tissue Eng Regen Med. 2017;11(12):3362-72; Brohlin M, etal., Characterisation of human mesenchymal stem cells following differentiation into Schwann cell-like cells. Neurosci Res. 2009;64(1):41-9; and Kingham PJ, et al., Adipose-derived stem cells differentiate into a Schwann cell phenotype and promote neurite outgrowth in vitro. Exp Neurol. 2007;207(2):267-74).

### Example 5 - in Vivo Testing of the Nerve Guides

The object of this example is to determine the ability of aligned DPC sheets to restore motor nerve function in a rat facial nerve defect. The ability of a nerve conduit prepared, e.g., according to Examples 1-4, to restore function in a nerve defect model in rats is assessed. A segmental nerve defect is created in the buccal branch of the rat facial nerve, and an engineered nerve conduit is used as a conduit to bridge the defect. Nerve regeneration is characterized histologically through the quantitative evaluation of host axon infiltration into the nerve conduit. Functional recovery is assessed through electrophysiology measurements. The engineered tissues may be compared to an autograft graft model and empty defects. It is expected that the scaffold-free DPCs constructs will provide both NTFs and guidance cues that will promote nerve regeneration at a similar or enhanced rate as autograft tissues.

Immunocompromised rats like RNU nude rats will be anesthetized using ketamine and xylazine via intraperitoneal injection. A preauricular incision will be made in the rat face with marginal bilateral mandibular extensions to expose the facial nerves. A length of 5 mm will be removed from the buccal branch of the facial nerve. This surgical procedure, followed by suturing a nerve conduit similar to those described in Example 1 and 2, was performed satisfactorily. We can have three experimental groups: 1) for positive control samples, following transection, the removed portion of the nerve will be rotated 180 degrees and sutured back to the nerve stumps to serve as an autograft; 2) for experimental groups, our scaffold-free nerve conduits generated using aligned DPC sheets will be sutured on either ends to the nerve stumps; and 3) as a negative control, the nerve gap will remain empty (6 animals/group. With 3 experimental groups, 6 animals/group, and 2 time points, 6 and 12 weeks post surgery). This defect model is a widely accepted method to assess facial nerve regeneration; furthermore, inverting and re-implanting the removed portion of the nerve is frequently used as a nerve autograft model.

Prior to euthanasia, electrophysiology measurements can be performed to determine restoration of nerve function. Following the euthanasia of the animals we can collect the engineered tissues and autografts for histological analyses. Longitudinal sections can be prepared to characterize axon extension across the engineered tissues or autografts. Immunostaining using antibodies against β-tubulin can be used to visualize the axons of host neurons. We can use image analysis software (imageJ software) to quantify the density of and extension of axons within the implanted tissue. T-tests (SPSS software) will be performed to distinguish differences in axon extension between the autograft and engineered tissues, significance can be determined at p<0.05.

We expect electrophysiology results to show that the implantation of SFCs into rat facial nerve defects to have similar or accelerated functional recovery as compared to autograft tissues. Furthermore, we expect that both of the autograft and engineered tissues will enable significant gain of function in comparison to the negative control. From our histological analyses, we expect that by 12 weeks, the axons from the host neurons will extend from the proximal to the distal ends of the implanted tissues and the autografts.

In further detail, the following protocols may be used.

Animal surgery and DPC sheet implantation: Immunocompromised rats aged 4-6 weeks may be purchased from Charles River Laboratories (USA) and are housed under standard conditions of alternate light and dark cycle. Before the surgical procedure, rats are anesthetized by intraperitoneal injection of ketamine hydrochloride (40 mg/kg) plus xylazine hydrochloride (5 mg/kg). The surgical site is prepared by trimming the hairs and cleaning with 10% povidone-lodine solution swab-sticks. Skin incision of 1 cm - 1.5 cm are placed over the buccal surface anterior to preauricular region and the buccal branch of the facial nerve is exposed. Using microscissors the nerve is separated and released from its underlying fascia and a section of the nerve is excised leaving two nerve stumps. The excised nerve is replaced by suturing a nerve conduit prepared essentially as described in Examples 1-4 to the remaining stumps. The conduit is secured with 9-0 nylon suture at the proximal and distal end of the excision. The skin incision is closed with 4-0 VICRYL® suture. After the surgery the rats are placed under the warm bag, allowed to recover consciousness and housed with access to food and water. Post-operative analgesia is administered by mixing acetaminophen (1.5 mg/ml) in the drinking water and the rats are observed for normal eating and drinking with active movement. The undamaged contralateral side serves as the positive control for this experiment and the experiment is carried out for up to 12 weeks with each group having 6 animals.

Histology and immunofluorescence analysis of the regenerated nerve: Four weeks after the surgery, rats are sacrificed by CO₂ inhalation and the regenerated nerves are explanted and fixed in 4% paraformaldehyde. The nerve tissue is flash frozen in OCT media and 5 micron sections are cut in cryostat. Routine hematoxylin and eosin staining is done to analyze the general architecture of the nerve.

Further sections are processed for immunofluorescence to detect the regenerated axons. Primary antibody for anti-beta tubulin (1:100) is incubated overnight at 40° C. and Alexa fluor 488 (1:500) is used as secondary antibody. Nuclei are counterstained by DAPI and the images were captured by NIKON eclipse TE2000-E microscope and processed with imageJ software.

### Example 6 - Treatment of Nerve Damage in a Patient

Autologous, allogeneic, or syngeneic tissue, such as DPCs, neural crest-derived tissue, adipose tissue, or bone marrow tissue, comprising mesenchymal stem cells or neural crest-derived stem cells, is cultured essentially as described in Examples 1-4, above, with suitable cell culture medium to form an over-confluent cell sheet and an aligned cylindrical nerve guide. The cell sheet is rolled into a cylindrical shape and is cultured to fuse the sheet into a cylindrical structure, essentially as described. The cylindrical structure is then washed, e.g., with normal saline or phosphate-buffered saline, and is then implanted in a subject at a site of nerve damage or defect, for example spanning a gap between damaged nerve endings. The nerve guide may be affixed in place by any compatible method, such as by suture or compatible glue. Any suitable surgical method may be used for implantation. As in Example 1, axon ingrowth into and through the cylindrical structure is expected, such that the nerve damage or deficiency is repaired. Two or more cylindrical nerve guides, e.g., as described in Example 2 and/or Example 3, may be linearly-aligned to bridge larger gaps between damaged nerve endings. Multiple implantations may also be utilized to bridge such larger gaps. The damaged or defective nerve may be a facial nerve.

While several examples and embodiments of the methods are described hereinabove in detail, other examples and embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A method of preparing a nerve conduit for use in repairing nerve damage or a nerve defect in a patient, comprising:
culturing a population of cells comprising a stem cell to confluence, or over-confluence, on a substrate comprising microgrooves aligned in a first direction, to produce a sheet of cells aligned in the first direction;
separating the aligned sheet of cells from the substrate;
rolling the sheet of cells in a second direction to form a roll of cells having a longitudinal axis extending in the first direction relative to the sheet of cells; and
culturing the roll of cells to fuse the rolled cell sheet to form a cylindrical cell structure, e.g., by adhesion and/or remodeling of adjacent layers of cells in the cylindrical roll of cells,
wherein the cells produce, or are induced or are differentiated to produce, a neurotrophic factor.

2. The method of claim 1, wherein the cells are obtained or derived from neural crest tissue and comprise neural crest stem cells.

3. The method of claim 1, wherein the cells are dental pulp cells or are obtained from a population of dental pulp cells.

4. The method of claim 1, wherein the cells produce one or more neurotrophic factors.

5. The method of claim 4, wherein the one or more neurotrophic factors is brain-derived growth factor (BDNF), glial-cell derived neurotrophic factor (GDNF), or neurotrophin-3 (NT-3).

6. (canceled)

7. The method of claim 1, further comprising, after the sheet of cells is produced, differentiating the cells to a cell type that produces one or more neurotrophic factors, such as a Schwann cell-like phenotype.

8. (canceled)

9. The method of claim 1, wherein the stem cell is a mesenchymal stem cell, an adipose-derived stem cell, an umbilical cord stem cell, a bone marrow stem cell, or an induced pluripotent stem cell.

10. (canceled)

11. (canceled)

12. The method of claim 1, wherein the microgrooved substrate comprises linear grooves oriented in the same direction or substantially in the same direction, or anisotropic grooves, such as parallel grooves and/or wherein the grooves of the microgrooved substrate are less than 250 microns (µ), less than 100 µ, less than 50 µ, less than 25 µ, or less than 10 µ in width, such as ranging from 1 µ to 10 µ in width.

13. (canceled)

14. A method of preparing a nerve conduit for use in repairing nerve damage or a nerve defect in a patient, comprising:
culturing a population of cells comprising a stem cell to confluence, or over-confluence, on a substrate, to produce a sheet of cells;
separating the sheet of cells from the substrate; and
culturing the sheet of cells anchored at two or more points to substantially align the cells in the sheet of cells,
wherein the cells produce, or are induced or differentiated to produce, a neurotrophic factor.

15. The method of claim 14, wherein, prior to culturing the anchored sheet of cells, rolling the sheet of cells to form a roll of cells, culturing the rolled sheet of cells anchored at two or more points along the length, e.g. along a longitudinal axis of the rolled sheet of cells, to fuse the rolled sheet of cells to form a cylindrical cell structure, e.g., by adhesion and/or remodeling of adjacent layers of cells in the cylindrical roll of cells, and to substantially align the cells in the rolled sheet of cells lengthwise in the rolled sheet of cells, or parallel to the longitudinal axis of the rolled sheet of cells.

16. The method of claim 14, wherein the cells are obtained or derived from neural crest tissue and comprise neural crest stem cells, and/or wherein the cells are dental pulp cells or are obtained from a population of dental pulp cells.

17. (canceled)

18. The method of claim 14, wherein the cells produce one or more neurotrophic factors.

19. The method of claim 18, wherein the one or more neurotrophic factors is brain-derived growth factor (BDNF), glial-cell derived neurotrophic factor (GDNF), or neurotrophin-3 (NT-3).

20. (canceled)

21. The method of claim 14, further comprising, after the sheet of cells is produced, differentiating the cells to a cell type that produces one or more neurotrophic factors, such as a Schwann cell-like phenotype.

22. (canceled)

23. The method of claim 14, wherein the stem cell is a mesenchymal stem cell, an adipose-derived stem cell, an umbilical cord stem cell, a bone marrow stem cell, or an induced pluripotent stem cell.

24. (canceled)

25. (canceled)

26. A fused, cylindrical cell structure prepared according to the method of claim 1.

27. A method of repairing nerve damage or a nerve defect in a patient, such as damage to a facial nerve or a facial nerve defect, comprising implanting the fused, cylindrical cell structure of claim 26 at a site of nerve damage or nerve defect in the patient.

28. (canceled)

29. A tissue culture vessel comprising a sheet of cells anchored at two or more points along its length in culture medium, wherein the sheet of cells is prepared by the method of claim 14.

30. A tissue culture vessel, comprising a fused, cylindrical cell structure prepared by claim 1.

31-39. (canceled)

40. A fused, cylindrical cell structure prepared according to the method of claim 14.
